(19) United States
(12) Patent Application Publication
(10) Pub. No.: US 2022/0389226 A1
(43) Pub. Date: Dec. 8, 2022

(54) A CONTINUOUS PROCESS FOR THE SYNTHESIS OF AZO DYES INVOLVING IN-SITU GENERATION OF DIAZONIUM SALTS

(71) Applicant: Council of Scientific and Industrial Research, New Delhi (IN)

(72) Inventors: Amol Arvind KULKARNI, Pune, Maharashtra (IN); Chinmay Abhay SHUKLA, Pune, Maharashtra (IN)

(21) Appl. No.: 17/776,870

(22) PCT Filed: Nov. 16, 2020
(86) PCT No.: PCT/IN2020/050961

§ 371 Date: May 13, 2022

(30) Foreign Application Priority Data
Nov. 15, 2019 (IN) 201911046583

(51) Int. Cl. C09B 67/00; C09B 67/54; C09B 41/00

(52) U.S. Cl. CPC C09B 68/24; C09B 67/0096; C09B 41/006

(57) ABSTRACT

The present disclosure provides a continuous process for the synthesis coupled compounds of diazonium salts including azo dyes involving in-situ generation of diazonium salts. It further discloses a set up to carry out the process for the synthesis of coupled compounds of diazonium salts.

### FIELD OF THE INVENTION

The present disclosure relates to a continuous process for the synthesis of azo dyes involving in-situ generation of diazonium salts. Particularly, the present disclosure provides a continuous process for the synthesis of diazonium compounds.

### BACKGROUND OF THE INVENTION

Azo compounds are traditionally synthesized in batch mode by reacting diazonium salt and coupling substrate viz. aniline, phenol, beta-naphthol, etc. Batch process has low productivity and significant wastewater generation due to frequent cleaning/washing of the reactor in every batch cycle.

Few reports exist for the continuous process for the synthesis of azo compounds and the synthesis of diazonium compounds. But such processes suffer from lack of industrial applicability and scalability.

The continuous process based on microreaction technology reported in the literature use extremely dilute conditions to avoid clogging. The reaction being one that generates a large amount of solids, clogging of reactors is a vexing issue, needing frequent cleaning and increases down time. The clogging of the reactor also results in an unresolved issue of improper and inadequate mixing of reactants, resulting in poor conversions of the reactants. This results in poor yields of the processes and makes them economically unattractive.

The article entitled “Exploring Flow Procedures for Diazonium Formation” by Ian R. Baxendale et. al and published in the journal “Molecules 2016, 21, 918; doi:10.3390/molecules21070918” reports a series of flow-based procedures to prepare diazonium salts for subsequent in-situ consumption. The article teaches the continuous flow process for the diazonium salt reactions and describes the use of different continuous reactor systems for the diazonium reactions. It also states that continuous reaction helps to control the reaction temperature and related side products. However, the conditions that the aforesaid article uses are very dilute and not suitable for practicing at large scale as a process. If the set-up described in this article is used for industrial concentrations of the reactants then the system will undergo clogging in a very short time. Therefore, the process does not help to carry out the flow synthesis at high concentrations.

Paul Watts et. al, in his article entitled “The in-situ generation and reactive quench of diazonium compounds in the synthesis of azo compounds in microreactors” published in the journal “Beilstein J. Org. Chem. 2016, 12, 1987-2004.” reports a micro-fluidic optimized process for the continuous flow synthesis of azo compounds. The article teaches continuous flow process for the formation of diazonium salt and in-situ azo coupling reaction with 2-naphthol to produce Sudan II azo dye in LTF-MS microreactor. It also describes a simple continuous flow set up consisting of T-mixers and PTFE tubing for the synthesis of naphtholic, phenolic and similar azo dyes like present invention. However, the conditions that the abovesaid article uses are very dilute and not suitable for practicing at large scale as a process. The set-up described in this article uses LTF glass micro reactors which are not suitable for handling of slurry when diazonium salt precipitates.

The processes known in the art have various issues as discussed above. It is therefore, the objective of the present disclosure to overcome the issues in the prior art.

### OBJECTS OF THE INVENTION

One objective of the present disclosure is to provide a continuous process for the synthesis of azo dyes involving in-situ generation of diazonium salts.

Another objective of the present disclosure is to provide an efficient continuous process for the synthesis of diazonium compounds.

Yet another objective of the present disclosure is to provide a continuous process for the synthesis of diazonium compounds without any unreacted aniline and without any impurities and maximum selectivity towards the desired azo compounds.

Further objective of the present disclosure is to provide a continuous process for the synthesis of diazonium compounds with >90% yield and 100% conversion.

### SUMMARY OF THE INVENTION

In an aspect of the present disclosure there is provided a continuous process for the synthesis of coupled compounds of diazonium salts, the process comprising: (a) reacting at least one primary amine with NaNO₂ and acid in a continuous reactor at pH<2 at a temperature in the range of 0° C.-5° C. for a period in the range of 0-10 min to form a diazonium salt, wherein said continuous reactor is selected from the group consisting of a gas-liquid bubble column reactor, air-loop lift reactor, a tubular reactor or a continuous stirred-tank reactor (CSTR); (b) reacting the diazonium salt as obtained in step (a) with at least one coupler in the presence of a base or buffer in a continuous bubble column reactor or air-loop lift reactor at a temperature in the range of 0° C.-25° C. and residence time up to 300 min and air flowrate in the range of 800 ml/min-1200 ml/min followed by filtering under vacuum to obtain the corresponding coupled compounds of diazonium salt.

In another aspect of the present disclosure, there is provided a set up to carry out the process for the synthesis of coupled compounds of diazonium salts of the present disclosure, the apparatus comprising: (i) Storage vessel for HCl solution (FIG. 1, 1); (ii) Storage vessel for sodium nitrite solution (FIG. 1, 2); (iii) storage vessel for aniline (FIG. 1, 3); (iv) Storage vessel for coupling substrate (FIG. 1, 4); (v) preheated or precooled process stream (FIG. 1, 5); (vi) reactor assembly for multistep diazotization and azo coupling reaction (FIG. 1, 6); (vii) filtration (FIG. 1, 7); (viii) waste tank (FIG. 1, 8), and (ix) outlet for diazo product (FIG. 1, 9).

### BRIEF DESCRIPTION OF THE DRAWINGS

The detailed description is described with reference to the accompanying figures. In the figures, the left-most digit(s) of a reference number identifies the figure in which the reference number first appears. The same numbers are used throughout the drawings to reference like features and components.

FIG. 1 represents process flow diagram for synthesis of azo compounds wherein (FIG. 1, 1) is Storage vessel for HCl solution, (FIG. 1, 2) is Storage vessel for sodium nitrite solution, (FIG. 1, 3) is storage vessel for aniline, (FIG. 1, 4) is Storage vessel for coupling substrate, (FIG. 1, 5) is preheated or precooled process stream, (FIG. 1, 6) is reactor assembly for multistep diazotization and azo coupling reaction (FIG. 1, 7) is filtration, (FIG. 1, 8) waste tank and (FIG. 1, 9) azo product outlet.

FIG. 2 represents NMR Spectra of Sudan I dye synthesized by Continuous process

FIG. 3 represents NMR Spectra of Solvent Yellow 16 dye synthesized by Continuous process.

FIG. 4 represents NMR Spectra of Solvent Red 24 dye by Continuous process.

FIG. 5 represents NMR Spectra of Solvent Yellow 56 dye by Batch experiment.

FIG. 6 (a-b) shows the diazotization and coupling reaction for the solvent red 24 dye.

FIG. 7 (a-b) shows the diazotization and coupling reaction for the solvent yellow 16 dye.

FIG. 8 (a-b) shows the diazotization and coupling reaction for the solvent yellow 56 dye.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions and compounds referred to or indicated in this specification, individually or collectively, and any and all combinations of any or more of such steps or features.

### Definitions

For convenience, before further description of the present disclosure, certain terms employed in the specification, and examples are collected here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The term “at least” refers to not less than or at a minimum. The term “at least one” refers to minimum one or more than one. The term “at least two” refers to minimum two or more than two.

The terms “comprise” and “comprising” are used in the inclusive, open sense, meaning that additional elements may be included. It is not intended to be construed as “consists of only”. Throughout this specification, unless the context requires otherwise the word “comprise”, and variations such as “comprises” and “comprising”, will be understood to imply the inclusion of a stated element or step or group of element or steps but not the exclusion of any other element or steps.

The term “including” is used to mean “including but not limited to”, “including” and “including but not limited to” are used interchangeably.

The term pH<2 as used herein refers to the pH value ranging between 0 to 2.

The phrase “other phenolic compounds” as used herein refers to any compound having hydroxylated aromatic ring, the hydroxy group being attached directly to the phenyl, substituted phenyl, or other aryl group.

The phrase “other anilines” as used herein refers to substituted anilines. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. The permissible substituents can be one or more and the same or different for appropriate organic compounds.

The phrase “solvent dyes” as used herein refers to dyes, such as Red 24 and its variant, Yellow 56 and its variant, Yellow 16 and its variant, and the like.

The phrase “residence time up to 300 min” as used herein refers to time varying from 0.2 to 300 min.

The term “base or buffer” as used herein refers to sodium hydroxide or sodium carbonate. However, other kinds of base or buffer can also be used which are known well to a person skilled in the art.

Unless defined otherwise, all technical and scientific terms used herein have the same meaning as commonly understood to one of ordinary skill in the art to which this disclosure belongs. Although methods and materials similar or equivalent to those described herein can be used in the practice or testing of the disclosure, the preferred methods, and materials are now described. All publications mentioned herein are incorporated herein by reference.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a temperature of about 0° C. to 25° C. should be interpreted to include not only the explicitly recited limits of about 0° C. and 25° C., but also to include sub-ranges, such as 5-20° C., 15-24° C., and so forth, as well as individual amounts, including fractional amounts, within the specified ranges, such as 1° C., 25.4° C., 25.44° C., for example.

The present disclosure is not to be limited in scope by the specific embodiments described herein, which are intended for the purposes of exemplification only. Functionally equivalent products, compositions, and methods are clearly within the scope of the disclosure, as described herein.

Azo dye coupling reaction in batch mode of operation traditionally involves the reaction between a primary amine with sodium nitrite in acidic conditions to form diazonium salts. This is a liquid-liquid/solid reaction, being an exothermic reaction, diazonium salts are formed which do not dissolve under reaction conditions and form a mass that precipitates out, making the handling of this solid a challenge. Being an exothermic reaction, diazonium salts can decompose generating nitrogen by-product and decreasing overall yield. In the second step of the process, the heat evolved in the exothermic first step degrades the diazonium salt formed evolving nitrogen and the solids formed clog the reactor, inhibiting the progress of the process. Prior arts overcome this clogging issue by using very dilute solutions. Alternately, micro channels or tubular reactors are proposed, but the issue of clogging is unresolved. To address this issue at hand, the present disclosure provides an efficient continuous synthesis approach for the synthesis of diazonium compounds including azo dyes to overcome the aforesaid issues which can be used for higher slurry concentration without clogging. Gas-liquid multiphase reactors like bubble column reactors and air-loop lift reactors as used in the process of the present disclosure offer good mixing of chemicals loaded in them in order to prepare azo dyes and diazonium compounds. The bubble column and air-loop reactors helps in overcoming the issue of clogging.

In an embodiment of the present disclosure, there is provided a continuous process for the synthesis of coupled compounds of diazonium salts, the process comprising: (a) reacting at least one primary amine with NaNO₂ and acid in a continuous reactor at pH <2 at a temperature in the range of 0° C.-5° C. for a period in the range of 0-10 min to form a diazonium salt, wherein said continuous reactor is selected from the group consisting of a gas-liquid bubble column reactor, air-loop lift reactor, a tubular reactor or a continuous stirred-tank reactor (CSTR); (b) reacting the diazonium salt as obtained in step (a) with at least one coupler in the presence of a base or buffer in a continuous bubble column reactor or air-loop lift reactor at a temperature in the range of 0° C.-25° C. and residence time up to 300 min and air flowrate in the range of 800 ml/min-1200 ml/min followed by filtering under vacuum to obtain the corresponding coupled compounds of diazonium salt.

In an embodiment of the present disclosure, there is provided a continuous process for the synthesis of coupled compounds of diazonium salts as described herein, wherein the coupled compounds of diazonium salts are selected from azo dyes selected from the group consisting of dispersive dyes, solvent dyes, acid dyes, reactive dyes, active pharmaceutical ingredients, and agro-chemicals that involve diazonium salts.

In an embodiment of the present disclosure, there is provided a continuous process for the synthesis of coupled compounds of diazonium salts as described herein, wherein the coupled compounds of diazonium salts are selected from the group consisting of Sudan I dye and its variants, solvent dyes and its variants.

In an embodiment of the present disclosure, there is provided a continuous process for the synthesis of coupled compounds of diazonium salts as described herein, wherein the at least one coupler is selected from the group consisting of beta-Naphthol, phenol, aniline pyrazoles or other phenolic compounds.

In an embodiment of the present disclosure, there is provided a continuous process for the synthesis of coupled compounds of diazonium salts as described herein, wherein the at least one primary amine is selected from aniline, 2-Aminoazotoluene, and other anilines.

In an embodiment of the present disclosure, there is provided a continuous process for the synthesis of coupled compounds of diazonium salts as described herein, wherein the at least one primary amine is selected from aniline and 2-Aminoazotoluene.

In an embodiment of the present disclosure, there is provided a continuous process for the synthesis of coupled compounds of diazonium salts as described herein, wherein the coupled compounds of diazonium salt have a yield in the range of 90 to 99%.

In an embodiment of the present disclosure, there is provided a continuous process for the synthesis of coupled compounds of diazonium salts as described herein, wherein conversion of the substrate is 100%.

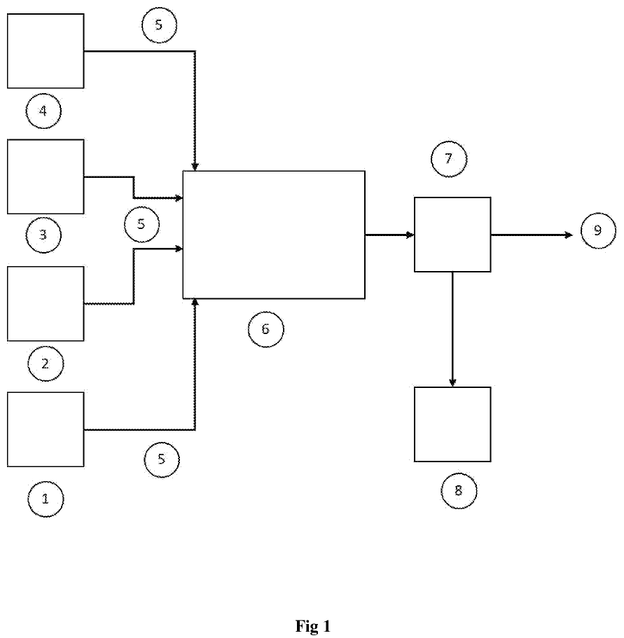

In an embodiment of the present disclosure, there is provided a set up to carry out the process for the synthesis of coupled compounds of diazonium salts, the process comprising: (a) reacting at least one primary amine with NaNO₂ and acid in a continuous reactor at pH<2 at a temperature in the range of 0° C.-5° C. for a period in the range of 0-10 min to form a diazonium salt, wherein said continuous reactor is selected from the group consisting of a gas-liquid bubble column reactor, air-loop lift reactor, a tubular reactor or a continuous stirred-tank reactor (CSTR); (b) reacting the diazonium salt as obtained in step (a) with at least one coupler in the presence of a base or buffer in a continuous bubble column reactor or air-loop lift reactor at a temperature in the range of 0° C.-25° C. and residence time up to 300 min and air flowrate in the range of 800 ml/min-1200 ml/min followed by filtering under vacuum to obtain the corresponding coupled compounds of diazonium salt, and the set up comprising: (i) Storage vessel for HCl solution (FIG. 1, 1); (ii) Storage vessel for sodium nitrite solution (FIG. 1, 2); (iii) storage vessel for aniline (FIG. 1, 3); (iv) Storage vessel for coupling substrate (FIG. 1, 4); (v) preheated or precooled process stream (FIG. 1, 5); (vi) reactor assembly for multistep diazotization and azo coupling reaction (FIG. 1, 6); (vii) filtration (FIG. 1, 7); (viii) waste tank (FIG. 1, 8), and (ix) outlet for diazo product (FIG. 1, 9).

In an embodiment of the present disclosure, there is provided a process for the synthesis of diazonium compounds, more particularly azo dyes comprising: (a) reacting a primary amine in the presence of an acid with sodium nitrite in a reactor selected from a gas-liquid bubble column reactor or air-loop lift reactor or a tubular reactor or a CSTR (continuous stirred-tank reactor) to form a diazonium salt solution; and (b) adding suitable coupling substrate selected from β-napthol, phenol, aniline or pyrazole to the mixture of step (a) in a bubble column reactor or air-loop lift reactor to form a suspension of the desire product followed by vacuum filtration to obtain the desired azo dye. The solids formed in step (a) are up to 20%, and the bubble column reactor used prevents agglomeration of the high concentration of solids by way of air sparging. The air sparging maintains a suspension and air sparging from bottom of the reactor pushes the solids out from the top. The FIG. 1 illustrates a process flow diagram for the synthesis of azo compounds. The process set up comprises of: (FIG. 1, 1) Storage vessel for HCl solution, (FIG. 1, 2) Storage vessel for sodium nitrite solution, (FIG. 1, 3) storage vessel for aniline, (FIG. 1, 4) Storage vessel for coupling substrate, (FIG. 1, 5) preheated or precooled process stream, (FIG. 1, 6) reactor assembly for multistep diazotization and azo coupling reaction (FIG. 1, 7) filtration, (FIG. 1, 8) waste tank and (FIG. 1, 9) outlet for diazo product. The process resolves the issues of clogging in the prior arts by using bubble column reactor, wherein gas generated in the process is used for mixing the high concentration of solids generated along with air sparging, thus avoiding clogging. The process is used for azo dyes selected from the group consisting of dispersive dyes, solvent dyes, acid dyes and reactive dyes, active pharmaceutical ingredients and agro chemicals that involve diazonium salts. The azo dyes are solvent dyes.

In another embodiment of the present disclosure, there is provided a continuous process for synthesizing coupled compounds of diazonium salts comprising: (a) reacting an amine (1 eq.) with NaNO₂ (1.05-1.2 eq.) and acid (3-4 eq.) in a continuous reactor at pH<2 and temperature in the range of 0° C.-5° C. for 0-10 min to form a diazonium salt; (b) reacting the diazonium salt with a coupler (1.05 eq) selected from beta-Naphthol, phenol, aniline or pyrazole in the presence of a base or buffer in a continuous bubble column reactor or air-loop lift reactor at a temperature in the range of 0° C.-25° C. and residence time up to 300 min and air flowrate in the range of 800 ml/min-1200 ml/min followed by filtering under vacuum to obtain the corresponding coupled compounds of diazonium salt. The reactor in step (a) is selected from air loop lift reactor, a tubular reactor or gas-liquid bubble column reactor or a CSTR (continuous stirred-tank reactor).

Although the subject matter has been described in considerable detail with reference to certain preferred embodiments thereof, other embodiments are possible.

### EXAMPLES

Following examples are given by way of illustration and therefore should not be construed to limit the scope of the present disclosure. The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may apply.

### Example: 1

### Batch Examples: Comparative Example

### Example 1: Synthesis Method for Solvent Red 24

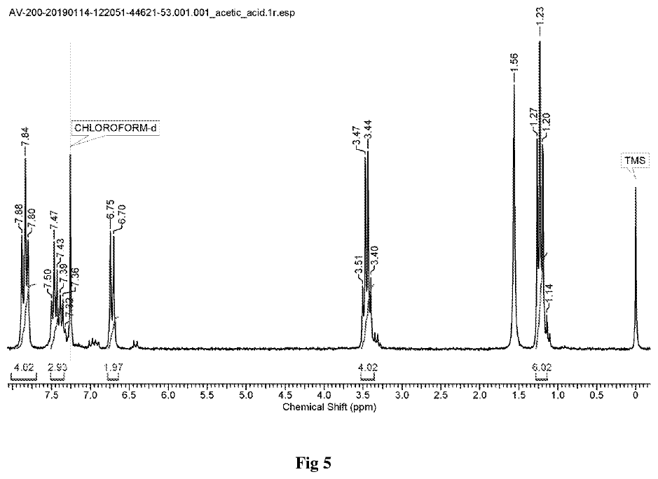

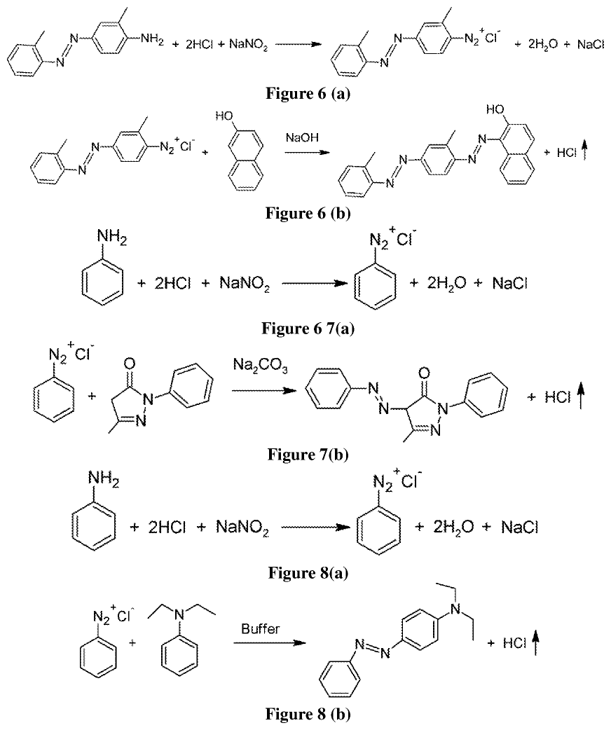

1 gm of 2-Aminoazotoluene was suspended in 10 mL of water in a jacketed batch reactor maintained at 5° C. 1.57 mL of 35% hydrochloric acid was added to the suspension and the solution was cooled to 5° C. 0.54 gm of sodium nitrite was dissolved in 5 mL of water and precooled to 5° C. This sodium nitrite solution was added to the reactor in a drop wise manner to maintain isothermal condition to generate diazonium salt. 0.67 gm of beta-naphthol and 0.83 gm of sodium hydroxide was dissolved in 15.6 mL of water and precooled and was added to diazonium salt solution. The reaction was complete in approximately 2 minutes. The product was filtered and dried. The isolated yield was in the range of 92%. FIG. 6(a-b) shows the diazotization and coupling reaction for the solvent red 24 dye.

### Example 2: Synthesis Method for Solvent Yellow 16

1 gm of aniline was dispersed in 5 mL water in a jacketed batch reactor maintained at 5° C. 2.86 mL of 35% hydrochloric acid was added to the suspension and the solution was cooled to 5° C. 0.97 gm of sodium nitrite was dissolved in 7 mL of water and precooled to 5° C. This sodium nitrite solution was added to the reactor in a drop wise manner to maintain isothermal condition to generate diazonium salt. 1.96 gm of 3-Methyl-1-phenyl-1H-pyrazol-5(4H)-one and 2.27 gm of sodium carbonate was dissolved in 32.5 mL of water and precooled and was added to diazonium salt solution. The reaction was complete in approximately 12 minutes. The product was filtered and dried. The isolated yield was in the range of 97%. FIG. 7(a-b) shows the diazotization and coupling reaction for the solvent yellow 16 dye.

### Example 3: Synthesis Method for Solvent Yellow 56

1 gm of aniline was suspended in 5 mL of water in a jacketed batch reactor maintained at 5° C. 2.86 mL of 35% hydrochloric acid was added to the suspension and the solution was cooled to 5° C. 0.97 gm of sodium nitrite was dissolved in 10 mL of water and precooled to 5° C. This sodium nitrite solution was added to the reactor in a drop wise manner to maintain isothermal condition to generate diazonium salt. 1.98 gm of diethylaniline, 7.92 gm of sodium acetate anhydrous and 0.16 gm of acetic acid were added in 14 mL of water. The suspension was precooled and was added to diazonium salt solution. The reaction was complete in approximately 4 hrs. The product was filtered and dried. The isolated yield was in the range of 91%. FIG. 8(a-b) shows the diazotization and coupling reaction for the solvent yellow 56 dye.

The process was conducted in batch mode and the yield of the dyes was in the range of 84-97%.

Continuous Flow Process with Bubble Column Reactor with Air

The continuous process conducted in bubble column reactor resulted in >90% yield. Present invention provides a clogging free process and a clean reactor and provides more than 90% yield of desired product, and 100% conversion of the limiting substrate. Present invention provides transformation of the batch process of synthesizing azo compounds into a continuous process by using a bubble column reactor for azo coupling reactions. The current inventions offer good yield and can handle higher slurry concentrations without clogging. Continuous vacuum filtration is also employed for separation. Further, the diazotization reaction step has also been performed in a CSTR or continuous jacketed tubular reactor.

### Example 4: Continuous Flow Synthesis of Sudan I Dye

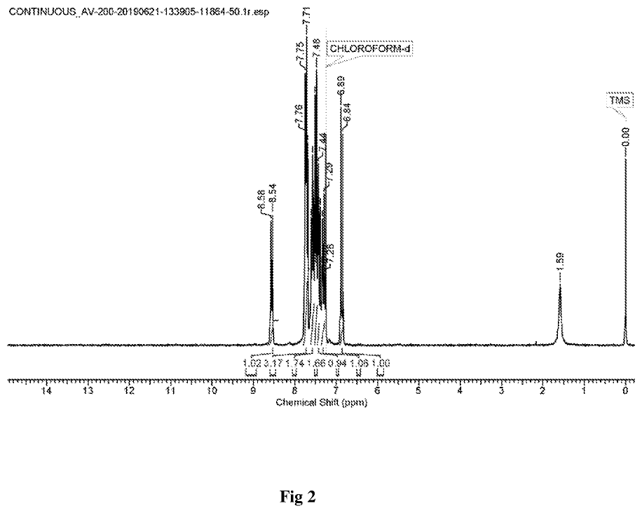

20 gm of aniline was suspended in 60 mL water and the solution was cooled below 5° C. 61.92 mL of 35% hydrochloric acid was slowly added to the suspension. 15.55 gm of sodium nitrite was dissolved in 40 mL of water. 32.51 gm of β-Naphthol and 25.76 gm of sodium hydroxide was dissolved in 606.75 mL of water. The stock solutions were precooled by ice or heat exchanger and peristaltic pump were used for passing the solutions. Aniline-HCl solution and sodium nitrite solution reacted in a jacketed tubular with residence time of 1 minute. The generated diazonium salt was further reacted with coupling substrate in a jacked bubble column reactor with residence time approximately 0.5 minutes. Utility temperature was maintained at 5° C. for both the reactors. The product dye was further isolated by vacuum filtration. The isolated yield was 92.04±3.50%. Conversion is 100%. NMR of the crude product is given in FIG. 2.

### Example 5: Continuous Flow Synthesis of Solvent Yellow 16 Dye

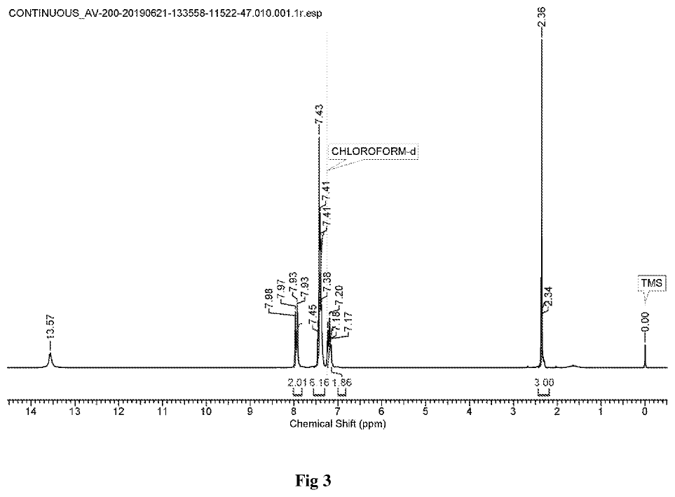

20 gm of aniline was suspended in 100 mL of water and the solution was cooled below 5° C. 57.16 mL of 35% hydrochloric acid was slowly added to the suspension. 15.55 gm of sodium nitrite was dissolved in 40 mL of water. 39.28 gm of 3-Methyl-1-phenyl-1H-pyrazol-5(4H)-one and 45.52 gm of sodium carbonate was dissolved in 1000 mL of water. The stock solution was precooled by ice or heat exchanger and peristaltic pump were used for passing the solutions. Aniline-HCl solution and sodium nitrite solution reacted in a jacketed tubular with residence time of 0.5 minutes. The generated diazonium salt was further reacted with coupling substrate in a jacked bubble column reactor with residence time less than 1 minutes. Utility temperature was maintained at 2° C. for both the reactors. The product dye was further isolated by vacuum filtration. The isolated yield was 96.60±2.18%. Conversion is 100%. NMR of the crude product is given in FIG. 3.

### Example 6: Continuous Flow Synthesis of Solvent Yellow 16 Dye

In example 5, diazotization was performed in a CSTR by directly adding liquid aniline, hydrochloric acid and sodium nitrite instead of preparing aniline hydrochloride salt separately. Yield was above 93%.

### Example 7: Continuous Flow Synthesis of Solvent Red 24 Dye

20 gm of 2-Aminoazotoluene was suspended in 200 mL of water and was cooled below 5° C. 31.5 mL of 35% hydrochloric acid was slowly added to the suspension. Magnetic stirrer was used to maintain the suspension throughout the experiment. 6.73 gm of sodium nitrite was dissolved in 10 mL water. 13.43 gm of β-Naphthol and 10.65 gm of sodium hydroxide was dissolved in 50 mL of water. The stock solutions were precooled by ice or heat exchanger and peristaltic pump were used for passing the solutions. 2-Aminoazotoluene —HCl solution and sodium nitrite solution reacted in a jacketed stirred tank reactor with residence time of ˜18 minutes. The generated diazonium salt was further reacted with coupling substrate in a jacked bubble column reactor with residence time approximately 2.5 minutes. Utility temperature was maintained at 2° C. for both the reactors. The product dye was further isolated by vacuum filtration. Yield was 92%. Conversion is 100%. NMR of the crude product is given in FIG. 4.

### Example 8 (Comparative): Continuous Flow Process with Bubble Column Reactor without Air

Example 4 was repeated without air sparging and clogging was observed within few minutes. The process was conducted devoid of air sparging in step b. Without air the process resulted in severe clogging of the reactor, as a result of which the reaction was not able to proceed further.

### Advantages of the Invention

The process of the present disclosure transforms the batch process of synthesizing azo compounds into a continuous process by using a bubble column reactor for azo coupling reactions. The present disclosure offers good yield and can handle higher slurry concentrations without clogging. Continuous vacuum filtration has been also employed for separation. Further, the diazotization reaction step has also been performed in a CSTR or continuous jacketed tubular reactor. Thus, the process of the present disclosure is: (a) scalable; (b) clogging free process and keeps the reactor clean; (c) column remains clean after entire process; (d) Higher throughput; (e) actual stoichiometric quantities used; (f) purity is maintained.

I/We claim:

1. A continuous process for the synthesis of coupled compounds of diazonium salts, the process comprising:
a) reacting at least one primary amine with NaNO₂ and acid in a continuous reactor at pH<2 at a temperature in the range of 0° C.-5° C. for a period in the range of 0-10 min to form a diazonium salt, wherein said continuous reactor is selected from the group consisting of a gas-liquid bubble column reactor, air-loop lift reactor, a tubular reactor or a continuous stirred-tank reactor (CSTR);
b) reacting the diazonium salt as obtained in step (a) with at least one coupler in the presence of a base or buffer in a continuous bubble column reactor or air-loop lift reactor at a temperature in the range of 0° C.-25° C. and residence time up to 300 min and air flowrate in the range of 800 ml/min-1200 ml/min followed by filtering under vacuum to obtain the corresponding coupled compounds of diazonium salt.

2. The process as claimed in claim 1, wherein coupled compounds of diazonium salts are selected from azo dyes selected from the group consisting of dispersive dyes, solvent dyes, acid dyes and reactive dyes, active pharmaceutical ingredients and agro chemicals that involve diazonium salts.

3. The process as claimed in claim 1, wherein the coupled compounds of diazonium salts are selected from the group consisting of Sudan I dye and its variants, solvent dyes and its variants.

4. The process as claimed in claim 1, wherein the at least one coupler is selected from the group consisting of beta-Naphthol, phenol, aniline pyrazoles or other phenolic compounds.

5. The process as claimed in claim 1, wherein the at least one primary amine is selected from aniline, 2-Aminoazotoluene, and other anilines.

6. The process as claimed in claim 1, wherein the coupled compounds of diazonium salt have a yield in the range of 90 to 99%.

7. The process as claimed in claim 1, wherein conversion of the substrate is 100%.

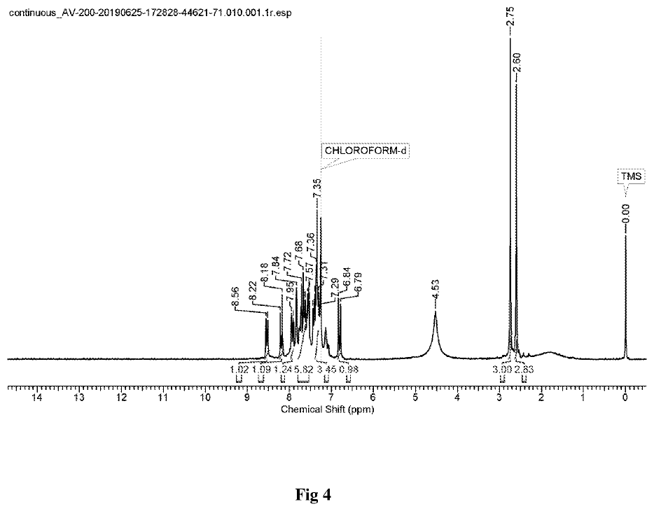

8. A set up to carry out the process for the synthesis of coupled compounds of diazonium salts as claimed in claim 1, the set up comprising:
i. Storage vessel for HCl solution (FIG. 1, 1);
ii. Storage vessel for sodium nitrite solution (FIG. 2, 2);
iii. storage vessel for aniline (FIG. 3, 3);
iv. Storage vessel for coupling substrate (FIG. 4, 4);
v. preheated or precooled process stream (FIG. 5, 5);
vi. reactor assembly for multistep diazotization and azo coupling reaction (FIG. 6, 6);
vii. filtration (FIG. 7, 7);
viii. waste tank (FIG. 8, 8), and
ix. outlet for diazo product (FIG. 9, 9).
